(19) United States
(12) Patent Application Publication
(10) Pub. No.: US 2017/0087544 A1
(43) Pub. Date: Mar. 30, 2017

(54) PROCESS FOR REMOVING CL FROM OXY-DEHYDRO CATALYST

(71) Applicant: UOP LLC, Des Plaines, IL (US)

(72) Inventors: J. W. Adriaan Sachtler, Des Plaines, IL (US); Bryan J. Egolf, Crystal Lake, IL (US); Rajeswar Gattupalli, Arlington Heights, IL (US)

(21) Appl. No.: 15/273,374

(22) Filed: Sep. 22, 2016

Related U.S. Application Data
(60) Provisional application No. 62/232,723, filed on Sep. 25, 2015.

(51) Int. Cl. B01J 38/06; B01J 23/96; B01J 23/58; C07C 5/48; B01J 23/42

(52) U.S. Cl. CPC B01J 38/06; C07C 5/48; B01J 23/42; B01J 23/58; B01J 23/96; C07C 2523/42; C07C 2523/58

(57) ABSTRACT

A process for the production of olefins from paraffins is presented. The process converts a paraffin stream through an oxy-dehydrogenation to process stream having olefins. The process includes a continuous catalyst regeneration system, where the catalyst cycles through the reactor and a regenerator. The process includes a treatment step for conditioning the catalyst to remove chloride on the catalyst after regeneration.

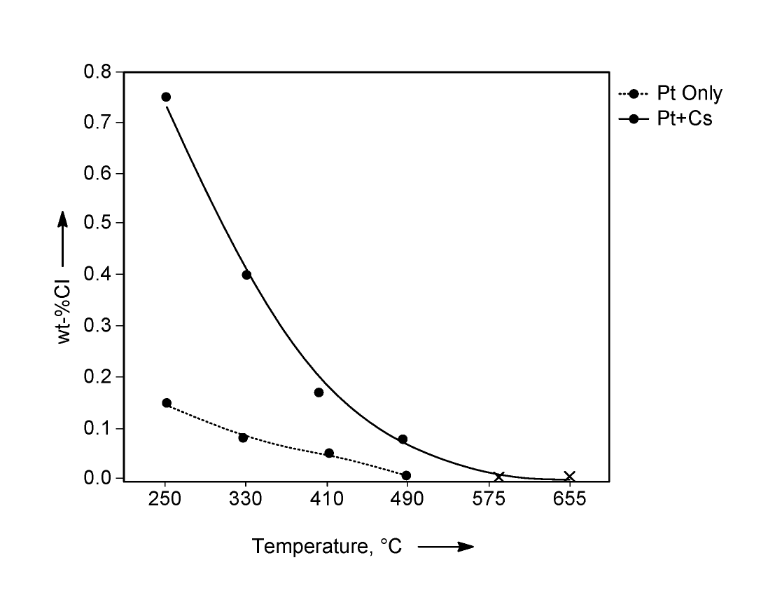

### CROSS-REFERENCE TO RELATED APPLICATION

This application claims priority from Provisional Application No. 62/232,723 filed Sep. 25, 2015, the contents of which are hereby incorporated by reference.

### FIELD OF THE INVENTION

The present invention relates to processes for the production of olefins using an oxidative dehydrogenation process.

### BACKGROUND

The use of plastics and rubbers is widespread in today's world. These plastics and rubbers are produced from the polymerization of monomers which are generally produced from petroleum. The monomers are generated by the breakdown of larger molecules to smaller molecules which are the precursors to plastics and rubbers. The monomers are then reacted to generate larger molecules comprising chains of the monomers. An important example of these monomers is light olefins, including ethylene and propylene, which represent a large portion of the worldwide demand in the petrochemical industry. Light olefins, and other monomers, are used in the production of numerous chemical products via polymerization, oligomerization, alkylation and other well-known chemical reactions. Producing large quantities of light olefin material in an economical manner, therefore, is a focus in the petrochemical industry. These monomers are essential building blocks for the modern petrochemical and chemical industries.

Light olefins, including propylene and butylenes, are primarily obtained through the cracking, either steam cracking or catalytic cracking, of heavier hydrocarbon streams, and in particular naphtha. With increasing demands, the sources are shifting, and propane and butane increasingly are generated from other sources, such as natural gas. The dehydrogenation of propane and butane provide for increasing the production of the desired olefins.

### SUMMARY

The present invention is a process for removing chloride from a catalyst to protect downstream processing equipment.

A first embodiment of the invention is a process for the regeneration of catalyst for an oxidative dehydrogenation reactor, comprising passing the catalyst, comprising a noble metal, to a catalyst regenerator to generate a regenerated catalyst stream; and passing the regenerated catalyst stream to a treatment unit to generate a regenerated stream comprising the catalyst with a chloride (Cl) content less than 1 wt % of the catalyst. An embodiment of the invention is one, any or all of prior embodiments in this paragraph up through the first embodiment in this paragraph further comprising passing steam to the treatment unit. An embodiment of the invention is one, any or all of prior embodiments in this paragraph up through the first embodiment in this paragraph wherein the treatment unit is operated at a temperature greater than 500° C. An embodiment of the invention is one, any or all of prior embodiments in this paragraph up through the first embodiment in this paragraph wherein the Cl content is reduced to less than 0.5 wt % of the catalyst. An embodiment of the invention is one, any or all of prior embodiments in this paragraph up through the first embodiment in this paragraph wherein the Cl content is reduced to less than 0.2 wt % of the catalyst. An embodiment of the invention is one, any or all of prior embodiments in this paragraph up through the first embodiment in this paragraph further comprising passing the regenerated catalyst stream to an oxidative dehydrogenation reactor, wherein the reactor is a moving bed reactor and generates a spent catalyst stream. An embodiment of the invention is one, any or all of prior embodiments in this paragraph up through the first embodiment in this paragraph further comprising passing the spent catalyst stream to the catalyst regenerator. An embodiment of the invention is one, any or all of prior embodiments in this paragraph up through the first embodiment in this paragraph wherein the catalyst regenerator comprises at least two parts, a first combustion section for removing carbon from the catalyst, and a second chlorination section for redispersing the noble metal on the catalyst. An embodiment of the invention is one, any or all of prior embodiments in this paragraph up through the first embodiment in this paragraph wherein the noble metal is platinum. An embodiment of the invention is one, any or all of prior embodiments in this paragraph up through the first embodiment in this paragraph wherein the catalyst comprises a noble metal, and at least one other metal selected from the group consisting of tin (Sn), germanium (Ge), lead (Pb), indium (In), gallium (Ga), thallium (Tl) and mixtures thereof. An embodiment of the invention is one, any or all of prior embodiments in this paragraph up through the first embodiment in this paragraph wherein the catalyst further comprises an alkali element. An embodiment of the invention is one, any or all of prior embodiments in this paragraph up through the first embodiment in this paragraph wherein the alkali element is selected from the group consisting of potassium (K), sodium (Na), cesium (Cs) and mixtures thereof. An embodiment of the invention is one, any or all of prior embodiments in this paragraph up through the first embodiment in this paragraph further comprising passing air to the treatment unit. An embodiment of the invention is one, any or all of prior embodiments in this paragraph up through the first embodiment in this paragraph further comprising passing hydrogen (H2), instead of passing air or following the passing of air, to the treatment unit.

A second embodiment of the invention is a process for the production of olefins comprising passing a paraffin stream, steam and an oxidizing agent to an oxidative dehydrogenation reactor, wherein the reactor is a moving bed reactor, comprising a catalyst with a noble metal, to generate a process stream comprising olefins, and wherein the moving bed reactor generates a spent catalyst stream; passing the spent catalyst stream to a catalyst regenerator to generate a regenerated catalyst stream; passing the regenerated catalyst stream to a treatment unit to generate a regenerated stream comprising catalyst with less than 1 wt % chloride content; and passing the regenerated stream to the oxidative dehydrogenation reactor. An embodiment of the invention is one, any or all of prior embodiments in this paragraph up through the second embodiment in this paragraph wherein the catalyst regenerator comprises at least two parts, a first combustion section for removing carbon from the catalyst, and a second chlorination section for redispersing the noble metal on the catalyst. An embodiment of the invention is one, any or all of prior embodiments in this paragraph up through the second embodiment in this paragraph wherein the Cl content is reduced to less than 0.5 wt % of the catalyst. An embodiment of the invention is one, any or all of prior embodiments in this paragraph up through the second embodiment in this paragraph further comprising passing steam to the treatment unit. An embodiment of the invention is one, any or all of prior embodiments in this paragraph up through the second embodiment in this paragraph wherein the treatment unit is operated at a temperature greater than 500° C. An embodiment of the invention is one, any or all of prior embodiments in this paragraph up through the second embodiment in this paragraph wherein the paraffin comprises propane or n-butane or isobutane.

Other objects, advantages and applications of the present invention will become apparent to those skilled in the art from the following detailed description and drawing.

### BRIEF DESCRIPTION OF THE DRAWING

The FIGURE shows the effect of temperature on the removal of chloride for catalyst comprising platinum, and for a catalyst comprising platinum and cesium.

### DETAILED DESCRIPTION

The present invention relates to the treatment of a catalyst used in an oxidative dehydrogenation process. The catalyst is in a continuous regeneration process in combination with the oxidative dehydrogenation reaction, wherein the catalyst is cycled through the oxidative dehydrogenation reactor and a catalyst regenerator. The oxidative dehydrogenation reactor can comprise one or more reactors. The regeneration process includes the use of a chlorination step for redistributing the catalytic metal over the support. However, this leads to a catalyst having a relatively high amount of chloride on the catalyst. In the oxidative dehydrogenation process, steam is added. The catalyst in the oxidative dehydrogenation reactor is thus exposed to steam. The steam leaches some of the chloride off the catalyst. This leaching of chloride leads to the production of hydrochloric acid (HCl) which is a corrosive acid. This is corrosive to the reactor and to downstream equipment. Treating the regenerated catalyst to remove chloride reduces the corrosion due to chloride stress corrosion and enhances equipment life. By adding a treatment unit to remove chloride from catalyst returning from the regenerator, one is protecting downstream equipment from corrosion which improves operating life of the downstream equipment, including the oxidative dehydrogenation reactor. Preliminary chloride targets provided by in-house metallurgy experts were with a desired range for commercial reactors in the range of 5-10 mol-ppm. This is estimated to correspond to a catalyst with a 0.032 wt % Cl content, based upon the operating conditions of the reactor.

The catalyst in the present invention is a noble metal catalyst on a support. The catalyst can also include promoter metals. The invention is part of a process for the regeneration of a catalyst used in an oxidative dehydrogenation reactor. The process includes passing the catalyst to a catalyst regenerator to generate a regenerated catalyst stream. A final step before passing the regenerated catalyst is treating the catalyst to reduce the chloride content. The regenerated catalyst is passed to a treatment unit for this final step before passing to the reactor. The treatment unit processes the catalyst to reduce the chloride content of the catalyst to less than 1 wt % of the catalyst.

In a preferred treatment, the catalyst is treated to reduce the chloride content to less than 0.5 wt % of the catalyst. In a more preferred treatment, the catalyst is treated to reduce the chloride content to less than 0.2 wt % of the catalyst. In a most preferred treatment, the chloride content is reduced to less than 0.05 wt % of the catalyst.

The regeneration process includes passing the catalyst to a regenerator where the accumulate coke on the catalyst is burned off in one or more combustion sections of the regenerator. The combustion section generates a catalyst with reduced coke content. Preferably substantially all the coke has been removed. During the combustion of the coke, the noble metal on the catalyst is subject to agglomeration. The regenerator further includes a chlorination section and the catalyst with reduced coke content is then passed to the chlorination section, where the noble metal on the catalyst is redispersed over the surface of the catalyst. The chlorination section includes the passing of a gas stream. The gas stream can comprise a composition of nitrogen and oxygen ranging from pure nitrogen to air, and a small amount of chlorine of chlorides. This generates a catalyst having chloride content that can be leached in the reactor.

The removal of the chloride is performed in a treatment unit, operated at conditions to remove the chloride. The process includes passing steam to the treatment unit, and the treatment unit is operated at a temperature greater than 250° C. with an appropriate time contacting the catalyst with steam. For reducing the chloride content further, the temperature can be raised to 400° C., and even further to 500° C. The temperature of operation is balanced with the level desired, and the contact time between the catalyst and steam in the treatment unit. This exposure can be varied by changing the steam content and the duration of the treatment. The treatment unit can further include the passing of either air or hydrogen to the treatment unit. While adding either air or hydrogen, but not both, to the treatment unit for chloride removal, the choice can depend on several factors. It was found that for an alkali containing catalyst, the use of steam and hydrogen while more effective in the removal of chloride, the removal of chloride needs to be balanced against limiting damage to the metals by the steam treatment. For alkali containing catalysts it was found that a steam/air mixture was more effective in most cases.

The catalyst for the present invention is a catalyst comprising a noble metal on a support. The noble metal comprises one or more of a Group VIII noble metal component, and includes platinum (Pt), iridium (Ir), rhodium (Rh), and palladium (Pd). A preferred noble metal is platinum. The catalyst can further include at least one other metal selected from tin (Sn), germanium (Ge), lead (Pb), indium (In), gallium (Ga) and thallium (Tl). The catalyst can also further include an alkali metal. A preferred alkali metal is sodium (Na), potassium (K) or cesium (Cs). The support can comprise an alumina, and a preferred alumina is theta-alumina, gamma-alumina, or a mixture of theta-alumina and gamma-alumina.

The process is a part of the dehydrogenation of paraffins, and can further include passing the regenerated and treated catalyst stream to an oxidative dehydrogenation reactor. A paraffin stream is passed to the oxidative dehydrogenation reactor to generate a process stream comprising olefins. The reactor is a moving bed reactor, and generates a spent catalyst stream, where the spent catalyst stream is passed to the regenerator. The preferred paraffin for dehydrogenation is a propane stream or a butane stream.

Chloride is removed by exposing the catalyst to steam at high temperatures. The FIGURE shows the results of chloride removal as a function of temperature for two catalysts. The catalyst was exposed to a 50% steam/air mixture for 20 hours. The first catalyst comprises platinum, and the second catalyst comprises platinum and cesium. The results show for temperatures greater than 500° C., most of the chloride is removed. While the information indicates that higher temperatures lead to more chloride removal, higher temperatures can damage the catalyst. The process for treating the catalyst is a balance of conditions that are within a window of temperatures and steaming conditions. Surprisingly it is found that it is much more difficult to chloride from the catalyst containing cesium. Therefore, prior art dealing with platinum on an alumina support without alkali is a much different system, and the operating conditions are different, and as such not applicable to the current invention.

Catalysts were prepared for experimental treatments. Following is a Table of results for prepared catalysts.

TABLE

| Description | Wt-% Pt only | Wt-% Pt + Cs | Wt-% Cl |
|---|---|---|---|
| Pt only | 0.56 | 0 | 0.64 |
| Pt + Cs | 0.56 | 3.04 | 1.38 |

While the invention has been described with what are presently considered the preferred embodiments, it is to be understood that the invention is not limited to the disclosed embodiments, but it is intended to cover various modifications and equivalent arrangements included within the scope of the appended claims.

1. A process for the regeneration of catalyst for an oxidative dehydrogenation reactor, comprising:
passing the catalyst, comprising a noble metal, to a catalyst regenerator to generate a regenerated catalyst stream; and
passing the regenerated catalyst stream to a treatment unit to generate a regenerated stream comprising the catalyst with a chloride (Cl) content less than 1 wt % of the catalyst.

2. The process of claim 1 further comprising passing steam to the treatment unit.

3. The process of claim 1 wherein the treatment unit is operated at a temperature greater than 250° C.

4. The process of claim 1 wherein the Cl content is reduced to less than 0.5 wt % of the catalyst.

5. The process of claim 4 wherein the Cl content is reduced to less than 0.2 wt % of the catalyst.

6. The process of claim 1 further comprising passing the regenerated catalyst stream to an oxidative dehydrogenation reactor, wherein the reactor is a moving bed reactor and generates a spent catalyst stream.

7. The process of claim 1 further comprising passing the spent catalyst stream to the catalyst regenerator.

8. The process of claim 1 wherein the catalyst regenerator comprises at least two parts, a first combustion section for removing carbon from the catalyst, and a second chlorination section for redispersing the noble metal on the catalyst.

9. The process of claim 1 wherein the noble metal is platinum, or palladium.

10. The process of claim 1 wherein the catalyst comprises a noble metal, and at least one other metal selected from the group consisting of tin (Sn), germanium (Ge), lead (Pb), indium (In), gallium (Ga), thallium (Tl) and mixtures thereof.

11. The process of claim 1 wherein the catalyst further comprises an alkali element.

12. The process of claim 11 wherein the alkali element is selected from the group consisting of potassium (K), sodium (Na), cesium (Cs) and mixtures thereof.

13. The process of claim 1 further comprising passing air to the treatment unit.

14. The process of claim 1 further comprising passing hydrogen (H2) to the treatment unit.

15. A process for the production of olefins comprising:
passing a paraffin stream, steam and an oxidizing agent to an oxidative dehydrogenation reactor, wherein the reactor is a moving bed reactor, comprising a catalyst with a noble metal, to generate a process stream comprising olefins, and wherein the moving bed reactor generates a spent catalyst stream;
passing the spent catalyst stream to a catalyst regenerator to generate a regenerated catalyst stream;
passing the regenerated catalyst stream to a treatment unit to generate a regenerated stream comprising catalyst with less than 1 wt % chloride content; and
passing the regenerated stream to the oxidative dehydrogenation reactor.

16. The process of claim 15 wherein the catalyst regenerator comprises at least two parts, a first combustion section for removing carbon from the catalyst, and a second chlorination section for redispersing the noble metal on the catalyst.

17. The process of claim 15 wherein the Cl content is reduced to less than 0.5 wt % of the catalyst.

18. The process of claim 15 further comprising passing steam to the treatment unit.

19. The process of claim 15 wherein the treatment unit is operated at a temperature greater than 500° C.

20. The process of claim 15 wherein the paraffin comprises propane, n-butane or isobutane.
